(19) United States
(12) Patent Application Publication
(10) Pub. No.: US 2025/0149154 A1
(43) Pub. Date: May 8, 2025

(54) MEDICAL SUPPORT SYSTEM, MEDICAL SUPPORT METHOD, AND NON-TRANSITORY RECORDING MEDIUM RECORDING MEDICAL SUPPORT PROGRAM

(71) Applicant: OLYMPUS MEDICAL SYSTEMS CORP., Tokyo (JP)

(72) Inventor: Kento IGUCHI, Tokyo (JP)

(73) Assignee: OLYMPUS MEDICAL SYSTEMS CORP., Tokyo (JP)

(21) Appl. No.: 18/928,368

(22) Filed: Oct. 28, 2024

Related U.S. Application Data
(60) Provisional application No. 63/546,960, filed on Nov. 2, 2023.

(51) Int. Cl. G16H 40/40

(52) U.S. Cl. CPC G16H 40/40

(57) ABSTRACT

A medical support system includes one or more processors comprising hardware, the one or more processors being configured to acquire support information comprising object information about plural objects used in an examination or a procedure, a loading parameter based on at least one of weight, size, or shape of the objects, container information about a container that moves while carrying the objects, and workflow information about a process of the examination or the procedure, and output arrangement information to load the objects into the container, based on the support information.

### RELATED APPLICATION DATA

This application is based on and claims priority under 35 U.S.C. § 119 to U.S. Provisional Application No. 63/546,960, filed on Nov. 2, 2023, the entire contents of which are incorporated herein by reference.

### BACKGROUND

### Field

The present disclosure relates to a medical support system for supporting loading of medical instruments onto a cart, a medical support method, and a non-transitory recording medium recording a medical support program.

### Description of Related Art

Medical instruments used in various medical actions at a medical facility are often stored at a location different from a location, such as an examination room, where a medical action is performed. In general, a medical instrument is carried from a storage space to an examination room or the like at the time of examination. For example, at the time of an endoscopic examination, equipment for the endoscopic examination is loaded onto a cart carrying an endoscope in a storeroom where the equipment is kept, and medical instruments such as the endoscope and the equipment are transported by the cart to an examination room.

Some procedure or some type of examination may involve many pieces of equipment (medical instruments). Depending on a capacity of a cart, medical instruments to be used by an operator may not all be loaded onto the cart, or movement of the cart may become unstable due to inappropriate loading. Moreover, a cart with a capacity that is larger than called for may sometimes be used to transport the medical instruments. In such a case, a large cart unnecessarily takes up a space in the examination room.

Accordingly, a system for supporting loading of medical instruments onto a cart with an appropriate capacity to be performed in an appropriate manner of loading is sought.

Note that Japanese Patent Application Laid-Open Publication No. 2001-236340 (hereinafter referred to as Patent Document 1) discloses a boxing procedure determination method for determining an appropriate arrangement position of a package or an object based on a feature value such as a size and by using a data structure having a tree-list structure. Furthermore, Publication No. 2023-000842 (hereinafter referred to as Patent Document 2) discloses a boxing support device for extracting an outline shape of a target object to be packed in a box by analyzing a target object image showing the target object, determining a non-occupied region where an object can be placed, by analyzing a box image showing the box, and determining an arrangement indicating recommended position and angle for placing the target object in the non-occupied region in the box.

### SUMMARY

A medical support system according to an aspect of the present disclosure includes one or more processors comprising hardware, the one or more processors are configured to acquire support information comprising object information about plural objects used in an examination or a procedure, a loading parameter based on at least one of weight, size, or shape of the objects, container information about a container that moves while carrying the objects, and workflow information about a process of the examination or the procedure, and output arrangement information to load the objects into the container, based on the support information.

A medical support method according to an aspect of the present disclosure includes: acquiring support information comprising object information about plural objects used in an examination or a procedure, a loading parameter based on at least one of weight, size, or shape of the objects, container information about a container that moves while carrying the objects, and workflow information about a process of the examination or the procedure; and outputting arrangement information to load the objects into the container, based on the support information.

A non-transitory recording medium recording a support program according to an aspect of the present disclosure that cause a computer to perform: acquiring support information comprising object information about plural objects used in an examination or a procedure, a loading parameter based on at least one of weight, size, or shape of the objects, container information about a container that moves while carrying the objects, and workflow information about a process of the examination or the procedure, and outputting arrangement information to load the objects into the container, based on the support information.

### BRIEF DESCRIPTION OF THE DRAWINGS

FIG. 1 is a block diagram showing a medical support system according to a first embodiment of the present disclosure.

FIG. 2 is an explanatory diagram showing an example of a table held in a database 13.

FIG. 3 is an explanatory diagram showing an example of an inference unit 14.

FIG. 4 is an explanatory diagram for describing an example of processing by a calculation unit 15.

FIG. 5 is a flowchart for describing an operation of the first embodiment.

FIG. 6 is an explanatory diagram showing an example of arrangement display.

FIG. 7 is an explanatory diagram showing an example of arrangement display.

FIG. 8 is an explanatory diagram showing an example of objects, such as medical instruments, having different shapes and sizes.

FIG. 9 is an explanatory diagram showing an example where a plurality of objects E1 to E6 in FIG. 8 are arranged in a container.

FIG. 10 is a flowchart showing a flow that is adopted by a medical support system according to a second embodiment of the present disclosure.

FIG. 11 is an explanatory diagram showing an example where the plurality of objects E1 to E6 in FIG. 8 are arranged in a container.

### DETAILED DESCRIPTION

Hereinafter, embodiments of the present disclosure will be described in detail with reference to the drawings.

### First Embodiment

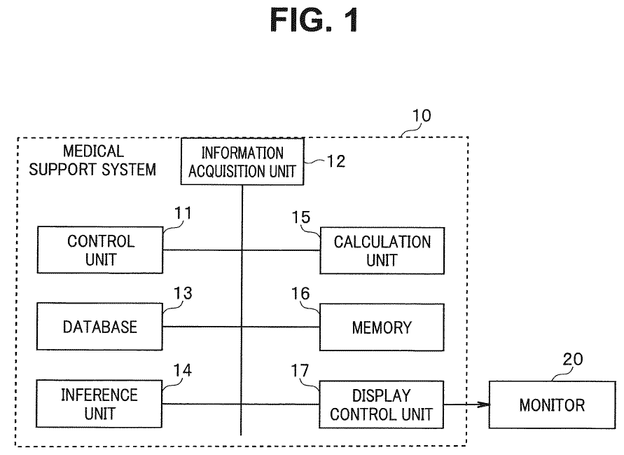

FIG. 1 is a block diagram showing a medical support system according to a first embodiment of the present disclosure.

The present embodiment is for supporting smooth transport of objects, such as medical instruments, by enabling presentation of an optimal loading method for objects at a time of loading the objects into a container (such as a cart) for transporting the objects including medical instruments. In the present embodiment, medical instruments including an endoscope are given as an example of medical instruments, but an endoscope may not be included in the medical instruments, and application to loading of objects including various medical instruments is possible.

Patent Documents 1 and 2 mentioned above disclose techniques for appropriately arranging packages at the time of boxing. However, Patent Documents 1 and 2 do not disclose techniques specialized for medical purposes, and do not give any disclosure or indication that takes into account the workflow of a medical action such as a procedure or an examination.

The present embodiment effectively supports preparation for a medical action by presenting an object loading method that takes into account workflow of the medical action. Note that the medical action in the specification is not limited to a medical action in a strict sense, and includes various tasks performed by a medical worker in association with a medical action such as an examination, a procedure, or a treatment.

A medical support system 10 in FIG. 1 supports loading of objects onto a container for transporting objects that are used in a medical action such as an examination, a procedure, or a treatment. Note that, as the container for transporting objects including medical instruments, a cart or a dedicated basket may be used, for example.

The medical support system 10 includes a control unit 11, an information acquisition unit 12, a database 13, an inference unit 14, a calculation unit 15, a memory 16, and a display control unit 17. Note that, as described later, with respect to the database 13, the inference unit 14, and the calculation unit 15, it suffices if the medical support system 10 includes at least one of the components.

The control unit 11 controls the entire medical support system 10 in an overall manner. The control unit 11 and each unit of the medical support system 10 may be configured by a processor that uses a CPU (central processing unit) or the like, and may operate according to a program stored in a memory, not shown, to control each unit, or at least some or all of functions may be implemented by an electronic circuit of hardware.

The information acquisition unit 12 is capable of receiving a signal from outside, and of outputting a signal from the control unit 11 to outside. For example, the information acquisition unit 12 may receive an operation signal or various pieces of data based on a user operation on an operation section such as a keyboard, not shown, and provide the same to the control unit 11. For example, the information acquisition unit 12 acquires information about objects, such as medical instruments, to be transported by a container (cart) and about a medical action such as an examination, a procedure, or a treatment that uses the objects (hereinafter, such information will be referred to as actual medical action information).

The database 13 configured by a memory includes a table including a plurality of data sets created in advance. Arrangement information is registered in the database 13 in relation to each medical action, the arrangement information indicating a manner of arrangement, in a container, of a plurality of objects, such as medical instruments, used in respective medical actions. For example, in the case where the medical action is an endoscopic examination, information indicating positions, in a container, where an endoscope and various pieces of equipment involved in the endoscopic examination, such as a cap to be attached to the endoscope, a treatment instrument, a guide wire, and a button for gas/liquid feeding, are to be arranged is registered in the database 13 as the arrangement information.

In a case where objects can be stacked and arranged in a container, information about an object to be arranged at each level may be registered in the database 13 as the arrangement information, for example. Alternatively, an order of loading objects into a container may be registered in the database 13 as the arrangement information. Alternatively, information indicating a position in a space inside a container where an object should be arranged may be registered as the arrangement information.

In the present embodiment, arrangement information for arranging each object at a position inside a container while taking into account workflow of a medical action is registered in the database 13. For example, arrangement information for arranging an object that is to be used in a medical action that is to be performed at a later timing near a bottom (first level) of a container, and for arranging an object that is to be used in a medical action that is to be performed at an earlier timing near a top (highest level) of the container is registered in the database 13.

For example, arrangement information for arranging an object that is to be used at a relatively early stage in a medical action, such as an examination or a treatment, close to a top in a container even if the object is rather heavy is registered in the database 13. Furthermore, arrangement information for collectively arranging a plurality of objects that are to be used together at a specific timing in a medical action, such as an examination or a treatment, at a specific position inside a container is registered in the database 13.

In general, an order of actions in a medical action, such as an examination, is set, and an order of using objects is also set. A medical action can be smoothly performed when an object that is to be used at an early timing is arranged on an upper side in a container and an object that is to be used at a later timing is arranged on a lower side in the container. Arrangement information is registered in the database 13 for each medical action, the arrangement information indicating an arrangement of each object in such a way that objects can easily be taken out from a container in an order according to the workflow of the medical action.

For example, in the case where the medical action is a duodenoscopic examination procedure, a procedure of inserting a guide wire in a lumen and inserting a treatment instrument along the inserted guide wire is performed. Accordingly, in such a case, arrangement information for loading the treatment instrument in a container first and then loading the guide wire above the loaded treatment instrument is registered in the database 13, for example. In other words, the arrangement information may be an arrangement pattern (optimized pattern), among arrangement patterns, that is based on a number of times of movement of objects at the time of taking out the objects from the container according to the workflow. For example, the arrangement information may be an optimized pattern according to which the number of times of movement of objects is the smallest. Note that in the case where a plurality of optimized patterns are obtained, the control unit 11 may set one of the optimized patterns as the arrangement information by setting a degree of priority described later.

The arrangement pattern here means any combination of a type or an inner dimension size of a container and an arrangement position of each object at the time of arranging a plurality of objects in the container. Furthermore, the optimized pattern means an arrangement pattern that takes the degree of priority or the like described later into account, among the arrangement patterns. Moreover, the arrangement information means the optimized pattern that is read out by the control unit 11.

Furthermore, an arrangement pattern (optimized pattern) that takes into account stability at the time of movement of the container may be registered in the database 13 as the arrangement information. For example, in relation to the arrangement information, one arrangement pattern among a plurality of arrangement patterns based on the number of times of movement of objects may be taken as the optimized pattern, based on a state of objects immediately after the objects are loaded into the container (start state) and a state of the objects after the container is moved at a predetermined speed from the start state (end state). Stability indicating a small change between the start state and the end state may be determined based on an experiment at a factory or human feedback from a user, or may be determined by physical simulation. In the case where there is a great change between the start state and the end state (low stability), the amount of movement of the objects loaded into the container is great, and there is a high possibility of an object falling from the container or positions inside the container getting changed.

When the amount of movement of objects from the start state to the end state is great, an object is possibly moved to a position where the object cannot be easily taken out, and thus, the amount of movement of objects can be small. An arrangement pattern (optimized pattern) with high stability where the amount of movement of objects from the start state to the end state is smaller than a threshold is registered in the database 13 as the arrangement information. For example, an optimized pattern with the smallest amount of movement of objects from the start state to the end state may be registered as the arrangement information. Moreover, in the case where there are a plurality of optimized patterns, all the optimized patterns may be presented to a user and the user may be made to select any one of the optimized patterns as the arrangement information, or the control unit 11 may select one optimized pattern as the arrangement information based on the degree of priority described later. Note that objects may be relatively stably transported when a cart with wheels is used as the container.

The control unit 11 refers to the database 13 based on the actual medical action information. The control unit 11 thus reads out the arrangement information for a medical action based on the actual medical action information. The control unit 11 outputs the arrangement information that is read out, to the display control unit 17.

The display control unit 17 generates, and outputs to a monitor 20, display data for displaying a display that is based on the arrangement information (hereinafter referred to as arrangement display). The monitor 20 displays the arrangement display on a display screen based on the display data that is inputted.

Note that the arrangement information may be registered in the database 13 in relation to each of a plurality of types of containers with different capacities or inner dimension sizes. For example, a medical worker operates the operation section, not shown, and inputs the actual medical action information, and also inputs information for specifying the container for transporting the objects (hereinafter referred to as container information). Note that the container information may be information about the inner dimension size of the container. The control unit 11 refers to the database 13 based on the actual medical action information and the container information. In other words, at least one piece of arrangement information that is about the container corresponding to the container information and that is about the objects and the medical action based on the actual medical action information is read out.

As described above, an optimized pattern is registered in the database 13 in relation to a medical action such as an examination or a procedure, by taking into account support information. The support information is comprising object information about two or more objects that are used in the medical action (hereinafter referred to as object information), information for specifying the container that moves while carrying the objects, the information being about the inner dimension size (the container information), and information about a process of the medical action (hereinafter referred to as workflow information), and the control unit 11 acquires, as the arrangement information, at least one optimized pattern based on the pieces of information. Note that the object information may include information about a parameter that is based on at least one of a weight, a size, or a shape of the object (hereinafter referred to as a loading parameter), for example.

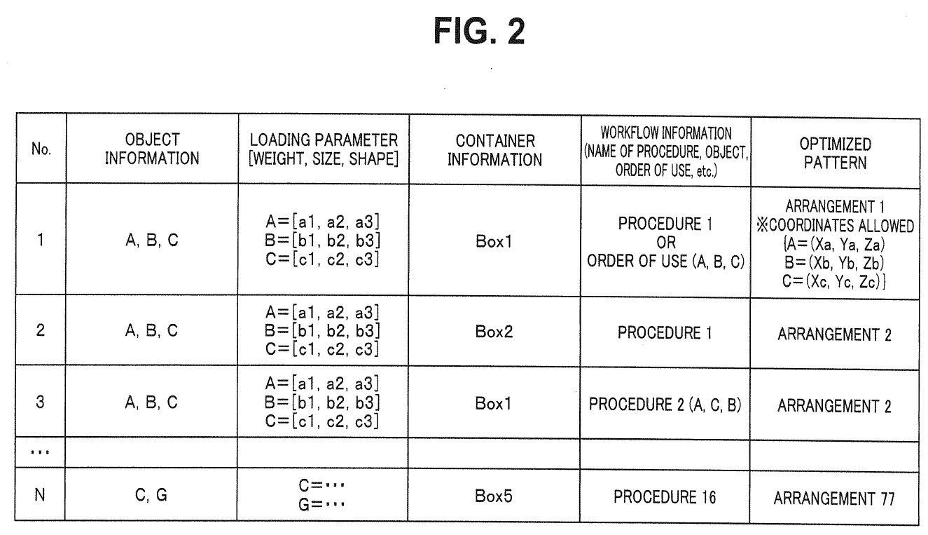

FIG. 2 is an explanatory diagram showing an example of a table held in the database 13. The example in FIG. 2 shows a table where information about an optimized pattern is registered in association with the object information, the loading parameter, the container information, and the workflow information. Note that the loading parameter includes information about weight, size, and shape, and the workflow information includes information about a name of a procedure, an order of using objects, and the like, for example.

Registered information of Nos. 1 and 2 is the information where the workflow information concerns a procedure 1. Objects to be used in the procedure 1 are objects A, B, and C, but registered information indicating that a container Box1 is used in No. 1 and a container Box2 is used in No. 2 is stored. In the case where the objects A, B, and C are to be transported using the container Box1 at the time of performing the procedure 1, the control unit 11 may refer to the database 13, and arrangement information indicating an arrangement 1 as the optimized pattern may be obtained. In the case where the objects A, B, and C are to be transported using the container Box2 at the time of performing the procedure 1, the control unit 11 may refer to the database 13, and arrangement information indicating an arrangement 2 as the optimized pattern may be obtained.

Note that coordinates in the container may be outputted as the optimized pattern. In the example in FIG. 2, it can be seen that, in relation to the arrangement 1, the object A may be arranged at coordinates (Xa, Ya, Za) in the container, the object B may be arranged at coordinates (Xb, Yb, Zb) in the container, and the object C may be arranged at coordinates (Xc, Yc, Zc) in the container.

The inference unit 14 includes an inference model for generating the arrangement information (hereinafter referred to as an arrangement inference model), and generates the arrangement information according to the medical action through execution of the arrangement inference model. The inference model to be used by the inference unit 14 is generated by generating, for a large number of medical actions, arrangement teaching information indicating how to arrange objects to be used in a medical action in a container according to the workflow of the medical action, and by performing machine learning of the generated arrangement teaching information. As described above, each data set in the database 13 includes the object information, the loading parameter, the container information, the workflow information, and at least one optimized pattern, and the control unit 11 acquires, as the arrangement information, the optimized pattern in a data set corresponding to the acquired object information, the acquired loading parameter, the acquired container information, and the acquired workflow information.

The control unit 11 supplies the actual medical action information to the inference unit 14. The inference unit 14 executes the arrangement inference model by using the actual medical action information, and generates the arrangement information for a medical action that is based on the actual medical action information. The generated arrangement information is supplied to the display control unit 17. The display control unit 17 causes the arrangement display based on the arrangement information determined by the inference unit 14 to be displayed on the display screen of the monitor 20.

Note that the inference unit 14 may include the arrangement inference model for each of a plurality of types of containers with different capacities or inner dimension sizes. In such a case, the inference unit 14 executes the arrangement inference model based on the actual medical action information and the container information, and thus obtains at least one piece of arrangement information that is about the container corresponding to the container information and that is for the medical action that is based on the actual medical action information.

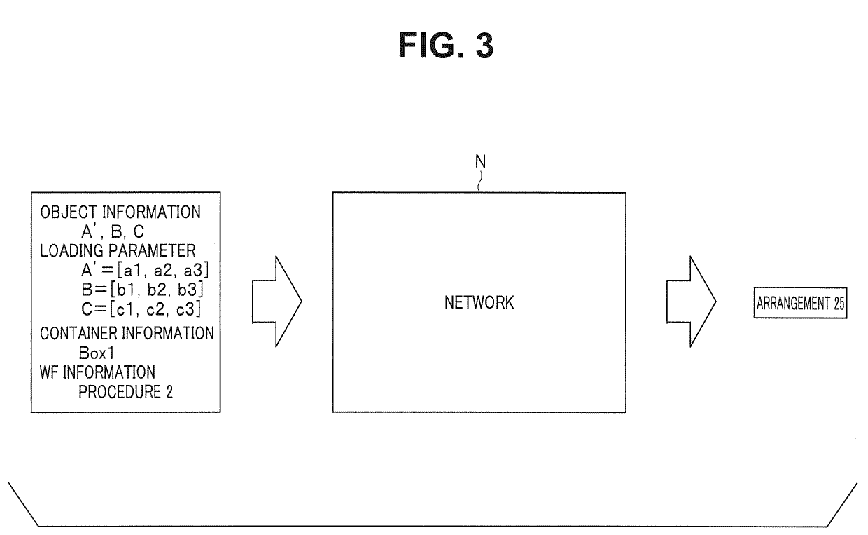

FIG. 3 is an explanatory diagram showing an example of the inference unit 14. The inference unit 14 may be configured by a network N for implementing the inference model, such as a neural network. A training device for the inference model may generate the inference model, for example, by taking the object information, the loading parameter, the container information, and the workflow information in FIG. 2 as an input, and by performing learning by using teacher data that takes the arrangement information in the table in FIG. 2 as correct answer information. Arrangement information that is the correct answer information is obtained by inputting the object information, the loading parameter, the container information, and the workflow information to the network N.

The example in FIG. 3 indicates that the object information indicating that the objects are A′, B, and C, the loading parameter regarding the objects A′, B, and C, the container information indicating that the container is Box1, and the workflow information indicating a procedure 2 are inputted to the network N, and that the arrangement information indicating an arrangement 25 is obtained as the correct answer information as a result from the network N.

The calculation unit 15 performs a calculation process for generating the arrangement information according to the workflow of the medical action by using various methods. More specifically, an optimal solution of a combinatorial optimization problem may be searched for. For example, the calculation unit 15 may generate the arrangement information by searching for a solution of an Ising model that takes, as an indicator for optimization, a matter that is to be prioritized at the time of loading medical instruments onto the cart (degree of priority). Note that various types of information involved in calculation by the calculation unit 15 are stored in the memory 16.

More specifically, the calculation unit 15 may determine the arrangement information by performing physics operation for when the container is moved while arranging objects in the container according to various arrangement patterns, and by evaluating stability as the indicator for optimization. Furthermore, the calculation unit 15 may take, as the arrangement information, a result of solving a traveling salesman problem through substitution of various arrangement patterns, the traveling salesman problem being for reducing the number of times of moving objects inside the container at the time of taking out the objects from the container during a medical action such as an examination/procedure.

The control unit 11 supplies the actual medical action information to the calculation unit 15. The calculation unit 15 performs calculation based on the actual medical action information, and generates arrangement information for a medical action that is based on the actual medical action information. The generated arrangement information is supplied to the display control unit 17. The display control unit 17 causes the arrangement display based on the arrangement information determined by the calculation unit 15 to be displayed on the display screen of the monitor 20.

Note that the calculation unit 15 may acquire information about the inner dimension size of a container corresponding to the container information from the memory 16 and perform calculation based on the actual medical action information and the information about the inner dimension size of the container, and may thus obtain at least one piece of arrangement information that is about the container corresponding to the container information and that is for the medical action that is based on the actual medical action information.

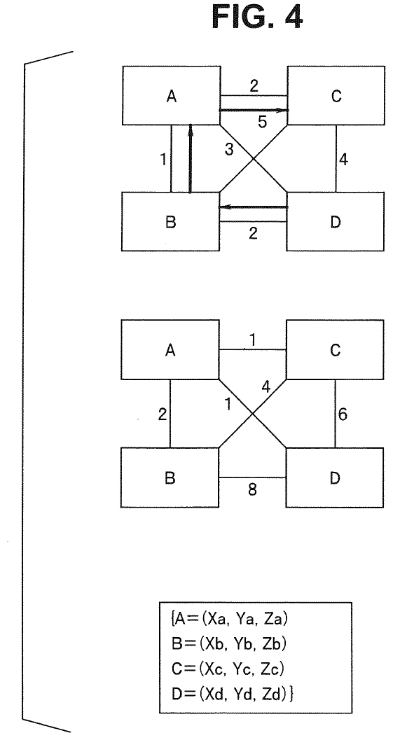

FIG. 4 is an explanatory diagram for describing an example of processing by the calculation unit 15. A top section in FIG. 4 shows an arrangement pattern where an object A is arranged on a first level, an object B is arranged at a front on a second level, an object C is arranged at a back on the second level, and an object D is arranged on a third level. A middle section in FIG. 4 shows an arrangement pattern where the object A is arranged on the first level, the object C is arranged at a front on the second level, the object B is arranged at a back on the second level, and the object D is arranged on the third level. Numbers attached to straight lines connecting respective objects indicate a level of labor that is involved at the time of taking out the objects in order (traveling). For example, in the diagram at the top section in FIG. 4, a value added to a straight line connecting the object A and the object C is 2, and thus, it is indicated that a level of labor that is involved to take out the object C after taking out the object A is 2. Note that, in the diagram, an interpretation that the level of labor at the time of taking out the object A after taking out the object C is also 2 is possible, but the level of labor cannot be 2, because, as described above, the object A and the object C are arranged on different levels. To avoid such an inconvenience, different cases may be assumed depending on an order of taking out, or a solution may be calculated based on a constraint, as described later.

A plurality of round routes are conceivable in relation to various arrangement patterns. Of the round routes, the calculation unit 15 calculates that a route along thick arrows does not involve much labor, and takes the route as an optimized pattern. A different optimized pattern may be calculated by using, as constraint conditions, constraints such as an arrangement pattern that is not permitted due to shapes of objects and a feasible traveling order, and such an optimized pattern may be outputted as the arrangement information. For example, the top section in FIG. 4 is an arrangement pattern where the object D is arranged on the third level, and thus, there is a physical constraint (i.e., not possible) with respect to the route indicated by the thick arrows according to which the object D is to be taken out first. In such a case, that the object D cannot be put first in the route is made a constraint condition, and such a solution is excluded and another optimal solution can be calculated. As a result, as shown in a bottom section in FIG. 4, arrangement information indicating coordinates, inside the container, where the objects A, B, and C are to be arranged can be obtained. Note that the so-called traveling salesman problem and specific solution methods for the constraint conditions have been examined in studies on various optimization problems and are known, and thus description of these methods is omitted herein.

Furthermore, a description is given above assuming that the workflow of a medical action based on the actual medical action information is known, but a case is also conceivable where a correspondence between a medical action and a workflow of the medical action is not clear. Accordingly, the information acquisition unit 12 may acquire the workflow information about the workflow of a medical action. In such a case, the control unit 11 supplies the actual medical action information and the workflow information to the database 13, the inference unit 14, and the calculation unit 15, and acquires the arrangement information.

Furthermore, a description is given above assuming that objects, such as medical instruments, used in a medical action based on the actual medical action information are known, but there is a case where a correspondence between a medical action and objects, such as medical instruments, used in the medical action is not clear. Accordingly, the information acquisition unit 12 may acquire the object information (including the loading parameter) about the objects, such as the medical instruments, to be loaded into the container. In such a case, the control unit 11 supplies the actual medical action information and the object information to the database 13, the inference unit 14, and the calculation unit 15, and acquires the arrangement information.

As described above, the database 13, the inference unit 14, and the calculation unit 15 are each capable of generating the arrangement information corresponding to the workflow of the medical action. Accordingly, as described above, it suffices if the medical support system 10 includes any one component among the database 13, the inference unit 14, and the calculation unit 15. Furthermore, in the case where the medical support system 10 is provided with the database 13, the inference unit 14, and the calculation unit 15, the control unit 11 may obtain the arrangement information by preferentially using the database 13, and by using the inference unit 14 in a case where there is a medical action that is not registered in the database 13, and by using the calculation unit 15 in a case where a score of an inference result from the inference unit 14 is relatively low.

Furthermore, in the case where the container information is not inputted, the control unit 11 may control each unit to generate a plurality of pieces of arrangement information corresponding to a plurality of usable containers, respectively. Note that a plurality of usable containers may be container information of containers (plural) used in the past, or may be container information that is registered in advance. Furthermore, in the case where a plurality of pieces of container information are inputted, a plurality of pieces of arrangement information corresponding to respective pieces of container information may be generated. Moreover, in the case where a plurality of optimized patterns are obtained, the control unit 11 may finally output one optimized pattern as the arrangement information by performing weighting and evaluation on the respective optimized patterns regarding whether the container is compact or not, a level of stability at the time of movement of the container, and a relationship with the workflow.

### (Checking of Loading)

For example, a reader for IC tags, bar codes or two-dimensional codes, or a camera may be attached to the container, and objects loaded into the container may be monitored. For example, a bar code reader may be attached to the container, and a user may load objects into the container while causing the reader to read bar codes attached to the objects. Furthermore, for example, a sensor, such as an IC tag, may be provided on an object to be loaded into a container or a wrapping of the object, and identification information of the object may be acquired at the time of loading of the object, and the control unit 11 may determine, based on the identification information, whether the object loaded into the container is included in the objects indicated by the object information.

Furthermore, for example, an image of objects, such as medical instruments, to be loaded into the container may be picked up, and a picked-up image may be acquired by the information acquisition unit 12, and the control unit 11 may perform image analysis to determine the objects that are loaded into the container. Note that in the case where checking of the objects loaded into the container is to be performed by using a camera, IC tags do not have to be attached to the objects.

Moreover, the control unit 11 may determine whether arrangement positions and a loading order of the objects loaded into the container match the arrangement positions and the loading order of the objects indicated by the arrangement information. In the case where the loading order is not normal, the control unit 11 can issue an alarm.

Furthermore, a dedicated preparation space may be provided before loading into a container is performed, and a reader for reading an IC tag that is present within a predetermined range on the preparation space may be provided. In such a case, IC tags on objects are automatically read when a container carrying the objects is placed on the preparation space. The control unit 11 may determine, for example, whether there is an object that is yet to be loaded, by determining a result of reading the IC tags, and may issue an alarm when there is an object that is yet to be loaded.

### (Operation)

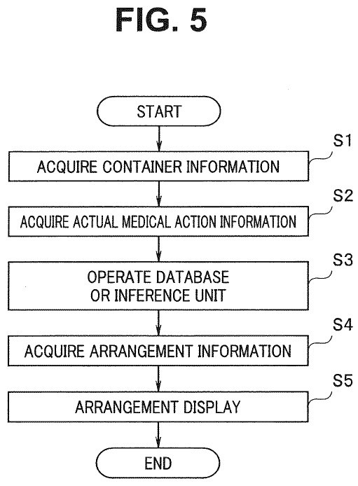

Next, an operation of the embodiment configured in the above manner will be described with reference to FIGS. 5 to 9. FIG. 5 is a flowchart for describing an operation of the first embodiment.

In steps S1 and S2 in FIG. 5, the information acquisition unit 12 acquires the container information and the actual medical action information. The control unit 11 refers to, for example, the database 13 by using the container information and the actual medical action information (S3). In other words, the control unit 11 searches and acquires, from the database 13, arrangement information for arranging, in the container indicated by the container information, objects that are to be used in performing the medical action indicated by the actual medical action information (S4). The control unit 11 outputs the arrangement information that is acquired as a result of search, to the display control unit 17.

Note that the description given above is an example where the arrangement information is acquired by using the database 13, but the arrangement information may alternatively be acquired by using the inference unit 14 or the calculation unit 15.

The display control unit 17 generates display data for arrangement display based on the arrangement information, and outputs the display data to the monitor 20. The arrangement display is thereby displayed on the display screen of the monitor 20 (S5).

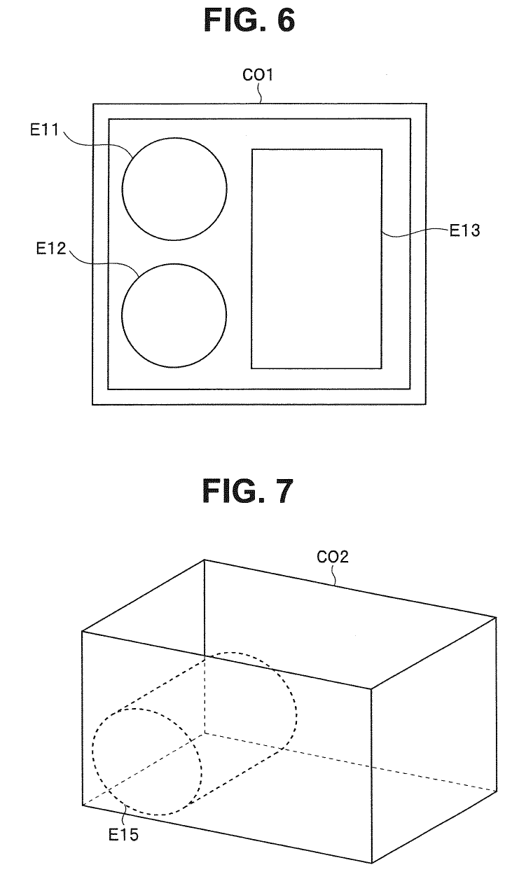

FIGS. 6 and 7 are explanatory diagrams showing examples of arrangement display. FIG. 6 shows an arrangement of objects when the entire container is seen from above the container. As the arrangement display, the display control unit 17 may perform animated display (hereinafter referred to as animated arrangement display) showing loading of objects in a sequential order. The example in FIG. 6 shows arrangement display for a first level, for example, in relation to animated arrangement display showing, level by level, objects that are to be sequentially loaded from a bottom of a container CO1. The example in FIG. 6 shows that objects E11 and E12 having a cylindrical shape and an object E13 having a rectangular parallelepiped shape may be arranged on the first level in the container, for example. The animated arrangement display displays, in a level-by-level manner, objects that are to be loaded.

FIG. 7 is an example of arrangement display based on a perspective view showing the container from obliquely above. FIG. 7 shows arrangement of objects on a per-object basis by showing the container in a see-through manner. The example in FIG. 7 shows that an object E15 having a cylindrical shape is first arranged inside a container CO2, and shows that the object E15 may be arranged at an end portion in a long side direction of the container CO2, with a height direction of the object E15 matching a short side direction of the container CO2.

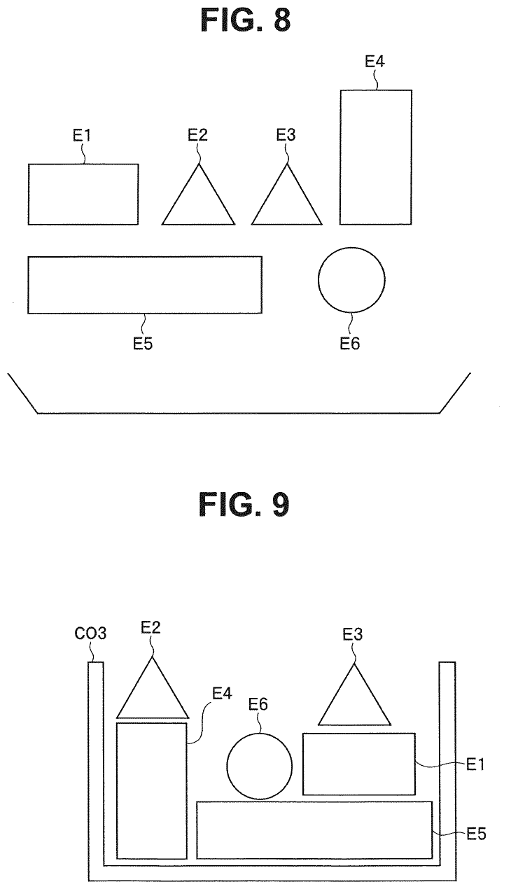

FIG. 8 is an explanatory diagram showing an example of objects, such as medical instruments, having different shapes and sizes. As objects to be transported, the example in FIG. 8 shows three objects E1, E4, E5 having rectangular parallelepiped shapes of different sizes, two objects E2 and E3 having a triangular pyramid shape, and one object E6 having a cylindrical shape. For example, the two objects E2 and E3 having a triangular pyramid shape are used at a relatively early timing in the workflow of a medical action, and the object E6 having a cylindrical shape is used next, and then, the objects E1, E4, E5 having rectangular parallelepiped shapes are used.

FIG. 9 is an explanatory diagram showing an example where the plurality of objects E1 to E6 in FIG. 8 are arranged in a container. For example, in the example in FIG. 9, in accordance with the workflow of the medical action, the objects E5 and E4 having rectangular parallelepiped shapes are arranged on a bottom side of the container, and then, the object E6 having a cylindrical shape and the object E1 having a rectangular parallelepiped shape are arranged at a higher position, and the two objects E2 and E3 having a triangular pyramid shape are arranged at a top level.

The objects E1 to E6 loaded into the container by such a loading method are carried into an examination room or the like. A medical worker uses the objects E2 and E3 having a triangular pyramid shape at a start of the workflow of the medical action, and then uses the object E6 having a cylindrical shape, and then uses the objects E1, E4, and E5 having rectangular parallelepiped shapes. In such a case, the objects E2, E3, E6, E1, E4, and E5 are each arranged from top to bottom of the container according to an order of use, and thus, the objects can be extremely easily taken out from the container without being frequently moved.

Note that the present embodiment describes an example where an optimal arrangement display suited to the workflow of a medical action is displayed by the monitor 20, but an optimal arrangement of objects may instead be presented by voice based on the arrangement information. Furthermore, in the case where loading is performed not by a person but by a loading robot, the arrangement information may be supplied to the loading robot without being presented through voice or an image. Note that the container may be a part of a robot.

As described above, in the present embodiment, arrangement display for arranging objects at positions in a container according to the workflow of a medical action is displayed at the time of loading of the objects into the container for transporting the objects, and smooth loading and transport of the objects can be supported.

### (Stock Management)

An object that is once loaded into a container for a medical action normally is to be disposed of or cleaned, and a stock in a storage space is reduced when loading is performed. Accordingly, stock management or a process of placing an order for an object may automatically be performed by using the reader for IC tags or the like described above.

When information about an object that is loaded is acquired from an IC tag reader or the like, the control unit 11 accesses a stock server managing a stock in the storage space, and outputs the acquired information about an object to the stock server.

The stock server includes a stock database holding information about all the objects stored in the storage space, and updates the stock database based on the information from the control unit 11. Furthermore, the stock server regularly monitors the stock database, and when the number of pieces of each type of object that are stored is smaller than the number of pieces of such object that should be in stock, the stock server places an order to a manufacturer, such as a maker, of the object so that the number of pieces of such object becomes equal to or greater than the number of pieces that should be in stock.

Accordingly, when a task of loading objects into a container to prepare for a medical action is performed, stock management and placement of order are automatically performed without a medical worker having to perform a burdensome management task regarding stock.

### Second Embodiment

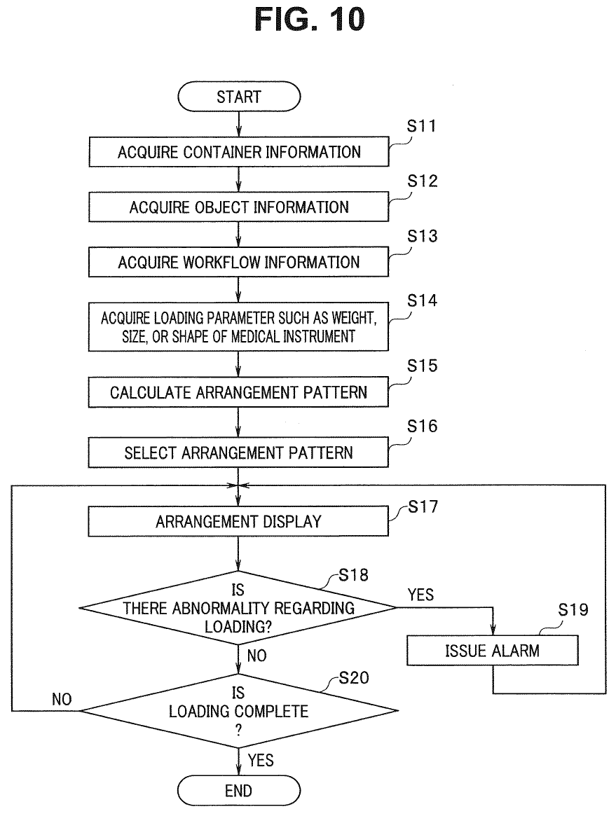

FIG. 10 is a flowchart showing a flow that is adopted by a medical support system according to a second embodiment of the present disclosure. A hardware configuration in the second embodiment is the same as a hardware configuration in the first embodiment. In the present embodiment, an optimal loading method can be presented at the time of loading of an object into a container, by taking into account at least one parameter (loading parameter) among weight, size, or shape of the object, a space in the container, and the like.

The present embodiment is different from the first embodiment with respect to registered contents in the database 13, the inference model of the inference unit 14, and calculation by the calculation unit 15.

Arrangement information that is registered in the database 13 in the second embodiment takes into account at least one of size or shape of an object. For example, in the database 13, an optimized pattern according to which a large object is arranged on a lower side in a container, a small object is arranged in a gap in the container, and an object not including a flat surface is arranged on an upper side in the container is registered in the arrangement information. Moreover, in the database 13, an optimized pattern that takes weights of objects into account and according to which a heavier object is arranged on a lower side in a container so that stability at the time of transport can be secured may be registered in the arrangement information.

Note that arrangement information for achieving an arrangement that takes into account the workflow of a medical action and that also takes into account weight, size, and shape of objects may be registered also in the database 13 of the second embodiment.

Furthermore, like the database 13, the inference unit 14 of the second embodiment adopts an inference model for generating arrangement information for achieving an arrangement where not only the workflow but also weight, size, and shape of objects are taken into account.

Moreover, like the database 13, the calculation unit 15 of the second embodiment acquires, through calculation, arrangement information for achieving an arrangement where not only the workflow but also weight, size, and shape of objects are taken into account.

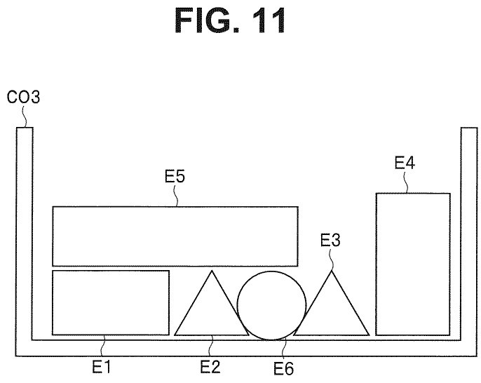

Next, an operation in the embodiment configured in the above manner will be described with reference to FIGS. 10 and 11. Note that FIG. 10 shows an example where the arrangement information is acquired by the calculation unit 15. Note that the database 13 and the inference unit 14 may be constructed to obtain the same arrangement information as the arrangement information that is acquired by the flow in FIG. 10, and the arrangement information may be acquired by using the database 13 or the inference unit 14.

In S11, S12, and S13 in FIG. 10, the information acquisition unit 12 acquires the container information, the object information, and the workflow information. Furthermore, in S14, the information acquisition unit 12 acquires the loading parameter such as the weight, the size, the shape or the like of objects based on the object information. Note that in the case where there are a plurality of objects for a same purpose with respect to an object in the object information, an object with a small size may be selected according to the object information. Furthermore, in the case where, with respect to an object in the object information, there are a plurality of objects that are for a same purpose but that are different in shape of wrappings, an object that achieves the best simulation result with respect to stability of the container at the time of loading may be selected according to the object information, for example.

The control unit 11 supplies the container information, the workflow information, and the loading parameter to the calculation unit 15. The calculation unit 15 determines an optimized pattern (arrangement information) that is an arrangement pattern indicating preferred arrangement positions of objects in the container, based on the container information, the workflow information, and the loading parameter.

For example, the calculation unit 15 obtains, as the arrangement information, an optimized pattern that is a preferred arrangement pattern of objects in the container, based on a following condition (i) based on the loading parameter or a following condition (ii) based on the workflow (S15).

### (i) Stable Arrangement

A heavy object is arranged on a lower side.
A flat plate-shaped object is arranged last (objects are prevented from falling out of the container by placing a flat plate-shaped object on the container as a lid).
An object that can easily fall out or that has a smooth surface is arranged in a gap.

### (ii) Arrangement Allowing Easy Removal

A heavy object is arranged on a lower side.
An object used first is arranged on top, although heavy.
Objects that are used at a same timing (workflow) are arranged near each other.

When the calculation unit 15 obtains the arrangement information according to the condition (i), stable movement can be performed when the container is moved to transport objects. Accordingly, an object may be prevented from getting damaged or the container may be prevented from rolling over due to falling of an object placed in the container or due to stacked objects collapsing.

When the calculation unit 15 obtains the arrangement information according to the condition (ii), objects are arranged in the container in an easily removable manner according to the workflow of the medical action, and the medical action may be smoothly performed.

A plurality of optimized patterns (arrangement information) are possibly calculated by the calculation unit 15. In such a case, the control unit 11 attaches degrees of priority indicating which of the conditions (i) and (ii) is to be prioritized, or attaches a degree of priority to each condition included in each of the conditions (i) and (ii), and selects one optimized pattern as the arrangement information based on the degrees of priority (S16). For example, one piece of arrangement information (optimized pattern) may be obtained based on a balance between compactness of arrangement, a level of stability, and the workflow. Note that the control unit 11 may present a plurality of pieces of arrangement information on the monitor 20, for example, to allow a medical worker to select one from the plurality of pieces of arrangement information.

The control unit 11 outputs one piece of arrangement information that is selected to the display control unit 17. The display control unit 17 displays the arrangement display based on the arrangement information, on the display screen of the monitor 20 (S17). The arrangement display may be animated arrangement display that sequentially shows arrangement of objects according to a loading order of the objects.

A medical worker loads objects into the container by referring to the arrangement display. An image of the manner of loading is picked up by an image pickup apparatus provided on the container, for example, and a picked-up image is acquired by the information acquisition unit 12 and supplied to the control unit 11. The control unit 11 performs image analysis of the picked-up image that is inputted, and thereby identifies an object that is loaded and determines whether loaded objects match the arrangement information, and determines whether there is an abnormality, such as wrong loading order, with respect to loading (S18). In the case where there is an abnormality, the control unit 11 issues an alarm in S19 by displaying the alarm on the monitor 20, for example, and returns to S17. In the case where there is no abnormality, the control unit 11 determines in S20 whether loading is complete or not. In the case where loading is not yet complete, the control unit 11 returns to S17, and when loading is complete, the control unit 11 ends the process.

FIG. 11 is an explanatory diagram showing an example where the plurality of objects E1 to E6 in FIG. 8 are arranged in a container. In the example in FIG. 11, the objects E1 and E4 that have rectangular parallelepiped shapes and that are relatively heavy are arranged on both ends on a bottom side of the container, and the objects E2, E5, and E6 that are relatively small and unstable are arranged in between, and the object E3 having a rectangular parallelepiped shape is arranged on the objects E1, E2, and E6.

As described above, in the present embodiment, an optimal loading method that increases stability during movement can be presented by taking into account not only the workflow of the medical action but also the weight and shape of objects, a space in the container, and the like.

The present disclosure is not limited to each embodiment described above, and can be embodied in the implementation phases while modifying structural elements without departing from the gist of the disclosure. Furthermore, various modes of disclosure may be implemented by combining as appropriate a plurality of structural elements disclosed in each embodiment described above. For example, several structural elements may be removed from all the structural elements disclosed in the embodiments. Moreover, structural elements of different embodiments may be combined as appropriate.

Moreover, of the techniques described herein, control and functions described mainly with reference to the flowcharts may be, in many cases, set by programs, and the control and the functions described above may be implemented by a computer reading and executing the programs. The programs may be entirely or partially recorded or stored, as a computer program product, in a removable medium such as a non-volatile memory, such as a flexible disk or a CD-ROM, a hard disk, or a storage medium such as a volatile memory, and may be distributed or provided at the time of shipping or via a removable medium or a communication line. A user may easily realize the medical support system of the present embodiments by downloading the programs via a communication network and installing the programs in a computer, or by installing the programs in a computer from a recording medium.

What is claimed is:

1. A medical support system comprising:
one or more processors comprising hardware, the one or more processors being configured to:
acquire support information comprising:
object information about plural objects used in an examination or a procedure,
a loading parameter based on at least one of weight, size, or shape of the objects,
container information about a container that moves while carrying the objects, and
workflow information about a process of the examination or the procedure; and
output arrangement information to load the objects into the container, based on the support information.

2. The medical support system according to claim 1, wherein the one or more processors are configured to output, as the arrangement information, at least one optimized pattern based on a number of times of movement of the objects at a time of taking out the objects from the container according to the process.

3. The medical support system according to claim 2, wherein the optimized pattern is an arrangement where the number of times of movement is smallest.

4. The medical support system according to claim 1, wherein the one or more processors are configured to output, as the arrangement information, at least one optimized pattern based on a start state being a state of arrangement immediately after the objects are loaded into the container and an end state being a state of arrangement after movement is performed at a predetermined speed from the start state.

5. The medical support system according to claim 4, wherein the at least one optimized pattern is calculated by physical simulation from the start state to the end state.

6. The medical support system according to claim 5, wherein the at least one optimized pattern is an arrangement that an amount of movement of the objects from the start state to the end state is smaller than a threshold.

7. The medical support system according to claim 1, wherein the container is a cart.

8. The medical support system according to claim 1, further comprising:
a memory storing a table including a plurality of data sets, each of the plurality of data sets including the support information and at least one optimized pattern, wherein
the one or more processors are configured to output, as the arrangement information, the at least one optimized pattern of the data set corresponding to the acquired object information, the acquired loading parameter, the acquired container information, and the acquired workflow information.

9. The medical support system according to claim 2, wherein the optimized pattern is calculated by an inference model obtained by learning an arrangement pattern when the objects in the object information are loaded into the container in the container information.

10. The medical support system according to claim 2, wherein the arrangement information is only one arrangement information outputted from the optimized pattern.

11. The medical support system according to claim 4, wherein the arrangement information is only one arrangement information outputted from the optimized pattern.

12. The medical support system according to claim 1, wherein, when two or more pieces of the container information are acquired, the one or more processors are configured to output the arrangement information for each piece of the container information.

13. The medical support system according to claim 1, wherein the one or more processors are configured to:
acquire identification information of the objects in the container, and
determine whether the identification information matches the object information included in the arrangement information.

14. The medical support system according to claim 1, wherein the one or more processors are configured to:
acquire an arrangement of the objects in the container, and determine whether the arrangement matches the arrangement information.

15. The medical support system according to claim 13, further comprising a sensor configured to acquire the identification information, wherein
the one or more processors are configured to be provided with the identification information acquired by the sensor, and identify the object loaded into the container.

16. A medical support method comprising:
acquiring support information comprising:
object information about plural objects used in an examination or a procedure,
a loading parameter based on at least one of weight, size, or shape of the objects,
container information about a container that moves while carrying the objects, and
workflow information about a process of the examination or the procedure; and
outputting arrangement information to load the objects into the container, based on the support information.

17. The medical support method according to claim 16, wherein the arrangement information is at least one optimized pattern based on a number of times of movement of the objects at a time of taking out the objects from the container according to the process.

18. The medical support method according to claim 16, wherein the arrangement information is at least one optimized pattern based on a start state being a state of arrangement immediately after the objects are loaded into the container and an end state being a state of arrangement after movement is performed at a predetermined speed from the start state.

19. The medical support method according to claim 16, further comprising:
acquiring identification information of the objects in the container, and
determining whether the identification information matches the object information included in the arrangement information.

20. A non-transitory recording medium recording a support program that cause a computer to perform:
acquiring support information comprising:
object information about plural objects used in an examination or a procedure,
a loading parameter based on at least one of weight, size, or shape of the objects,
container information about a container that moves while carrying the objects, and
workflow information about a process of the examination or the procedure; and
outputting arrangement information to load the objects into the container, based on the support information.
